(19) United States
(12) Patent Application Publication
(10) Pub. No.: US 2023/0338182 A1
(43) Pub. Date: Oct. 26, 2023

(54) ORTHOPEDIC SUPPORT CUSHION SYSTEM

(71) Applicants: PRESTON NELSON, MOUNT VERNON, IL (US); GEORGE A. PALETTA, ST. LOUIS, MO (US)

(72) Inventors: PRESTON NELSON, MOUNT VERNON, IL (US); GEORGE A. PALETTA, ST. LOUIS, MO (US)

(21) Appl. No.: 18/136,718

(22) Filed: Apr. 19, 2023

Related U.S. Application Data
(60) Provisional application No. 63/372,999, filed on Apr. 20, 2022.

(51) Int. Cl. A61F 5/30

(52) U.S. Cl. CPC A61F 5/30

(57) ABSTRACT

A combination of cushioning devices, as pillows or cushions, that have a surface area of one component of a hook and loop fastening device, and have applied to it a strap of the other component of a hook and loop fastening device, to allow the various pillow(s) to be applied to the limb of a patient during convalescence, or the pillows to be interconnected and stacked together, when used by the patient when resting, or sleeping, for ideal posture and positioning, during such convalescence.

### CROSS REFERENCE TO RELATED PATENT APPLICATIONS

This patent application claims the benefit of the priority date of provisional patent application Ser. No. 63/372,999, filed on Apr. 20, 2022, which is incorporated in its entirety herein.

### FIELD OF THE INVENTION

The concept of this invention is to provide a pillowed or cushion support system to support a body part during rest and rehabilitation, and during convalescence, in order to provide comfort to the healing body part, support during said healing process, for the enhancing health and benefit of the patient. A pillowed or a cushion support system includes a pillow or cushion, upon its outward surface having the application of one part of a hook and loop connecting system, while a strap like member carries the other part of the hook and loop system, so that the one or more straps may be applied to the pillow or cushion around the limb or body part of the patient during the healing process.

### BACKGROUND OF THE INVENTION

Obviously, pillows and cushions have long been used to function as a comfortable support for the user, which in this instance, is to add to the comfort of the patient during a healing procedure.

Such pillows and cushions are widely used as an adjunct object for humans or other species, utilized for comfortably and ergonomically positioning about certain of the parts of the body for longer term positioning as during rest, and during sleep, as known and used for eons.

There have been specially designed orthopedic pillows for support to specific body parts, such as wedges, cervical pillows, and even extraordinarily large pillows which encompass the entire body, to add to comfort during the healing process. To the applicant's knowledge, there have been no devices or system that has existed that allows one or more of smaller pillows capable of affixing to certain body parts to customizably adhere to the healing body part, such as an arm, or a leg, and other anatomy, which are recommended to remain immobilized following a surgical procedure, and during the long term of convalescence.

There are a variety of patents that relate to related technology, which can be seen in U.S. Pat. No. 6,957,462, that shows a pillow with a slidable strap through it, and clasp, for use for attachment to a portion of a user's body, and primarily used as a headrest. The published application to Moss, number U.S. 2013/0117939, shows a travel pillow for arm securement to rest against the head of the user.

Other publications show a pillow for application by straps or bandages to the foot of the user. Other publications show the use of medical bandages for holding a patient's broken arm against the body.

In addition, U.S. Pat No. 6,874,183, shows a form of S shaped pillow for flexible body support, along the length of the user, as previously referred to in this background.

### SUMMARY OF THE INVENTION

The broad concept of this invention is to provide the application of an orthopedic form of pillow like structures to certain body parts, during their healing process following surgery, or the corrections made to a broken bone, usually which entails many weeks of rest, to ease the extent of the convalescing process, during healing. For example, a broken arm may take six to eight weeks to recover back to a normal state after setting, and during that time, the patient must exercise significant caution in protecting the injured part, whether it be an arm, leg, ankle, foot, or the like, in order to achieve complete healing. In this particular instance, the more specific aspects of this invention include an especially structured pillow or cushion, one that is sized to conveniently fit contiguously with the injured body part, and then being held in position by means of cooperating straps, that may secure with the pillow by means of the hook and loop fastening means, otherwise known as the Velcro brand type fastening means, in order to add to the stability of the healing part, or to add to the patient's rest and comfort during the convalescing. Obviously, other means for fastening may be utilized, such as the strap may be formed of plastic, and stitched in place upon the surface of the pillow and may be resilient, to allow it to be expanded, for fitting of the specified body part therein, during its usage and application.

It is therefore, the principal object of this invention to provide a customizable orthopedic support system, including pillows or cushions and straps, designed for use with specific body parts during a healing process, for the usage, convenience and comfort of the patient.

Another object of this invention is to provide for the secure fit of a customized pillow or cushion through the use of a variety of fastening means, such as specially designed straps, that adhere the pillow or cushion to the vulnerable appendage or other body part of the patient during convalescence.

Another object of this invention includes the application of a number of pillows or cushions, that may be secured together, also by hook and loop fastening means, and then held by specialized straps to a specific patient's limb or body part while undertaking a healing process, such as a broken leg, arm, back or hip surgery, or the like.

A further purpose of this invention is to provide for the patient and assurance of comfort and support, to provide the patient with peace of mind as during resting, or sleep, so that the patient need not worry about the fragile body part being displaced during such periods of convalescence, or during associated sleep, throughout the prolonged healing process.

Another object of this invention is to provide a pillow or cushion means that can aid in the recovery process for a patient following surgery, on a particular body part, to which the pillow or cushion is applied during usage.

These and other objects may become more apparent to those skilled in the art upon review of the Summary of the Invention provided herein, and upon undertaking a study of the Description of its Preferred Embodiments, in view of the Drawings.

### BRIEF DESCRIPTION OF THE DRAWINGS

FIG. 1 shows the comfort pillow or cushion applied to a broken arm, through the use of associated straps, even while the patient is asleep, to assure comfort and support for healing of the limb.

FIG. 2 shows the components of the pillow or cushion assembly of FIG. 1, including its various straps, in which, in this instance, the straps are held to the pillow or cushion by the shown hook and loop fastening.

FIG. 3 discloses how two pillows or cushions may be joined together for use of application such as a leg cradle, during convalescence.

FIG. 4 shows the application of one or more pillows or cushions, by use of the associated straps, applied to a broken leg of the patient during the healing process.

FIG. 5 shows the plurality of pillows or cushions connected together with the hook and loop fastening means to apply along the length of the body portion of the patient, during convalescence, such as after experiencing back surgery.

### DESCRIPTION OF THE PREFERRED EMBODIMENTS

The concept of this invention, as previously summarized, is to provide an extended term cushioned support for the body, or an appendage, during rest and convalescence, as during sleep, and to prevent any unwanted impacting or displacement, or even shifting of the affected limb, during typical moments of rest or sleep. The object of the invention is to cushion the area that has been repaired, whether it be a broken limb, back surgery, surgery anywhere else on the body, as on the hip, where cushioning is desired to protect the healing area, and to allow its sustained healing, without any disruption, as can be understood.

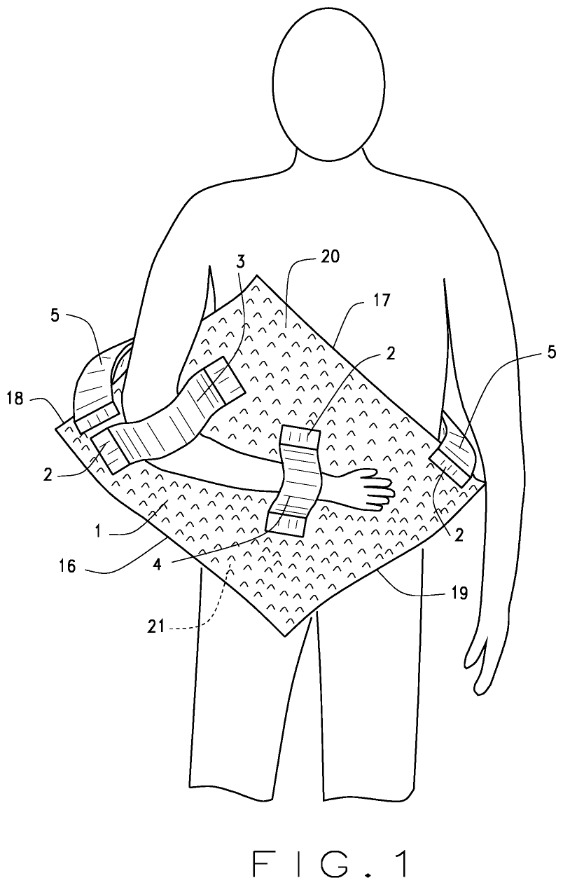

As can be seen in FIG. 1, it provides a view of the patient, when he is healing from a broken arm, as a forearm, the bicep region, or an elbow, or is recovering from surgery at these locations, and discloses how the pillow 1 having a surface configuration as a part of hook and loop type fasteners, as at 2, applied to the surface of the pillow, and the various straps, as at 3 and 4, may be wrapped around the arm at various locations, and applied and secured by the second part of the hook and loop type fasteners to the surface of the pillow 1, in order to secure the pillow in place, upon the arm during its healing process. The straps may be somewhat elastic, and have flexibility, so as to bias lightly upon the contiguous arm surfaces, during usage. And, where the straps are applied to the surface of the pillow or cushion, the straps 3, 4 are secured by one or the other hook and loop portion of the fastening means, or may be held by any equivalent type of fastener, to the surface of the pillow, or even a double sided adhesive tape may secure that portion of the fastener, to the surface of the pillow or cushion, and at the proper and convenient location. As noted, the patient may be undertaking sleeping, at this time, or as during resting, and has the pillow both supporting the affected arm, and adding cushioning to the healing area, during such rests and sleep, as noted herein. Obviously, such a cushioning means, or the application of the pillow may be applied even during the working hours, just to add to the comfort of the patient, and the affected arm, as can be understood.

It can be seen that there is a back strap, as at 5, that is also applied by the hook and loop fastening means, to the pillow, as noted, and can locate around the back of the patient, in order to provide for full securement of the pillow, both to the patient, and to the patient's arm, during convalescence.

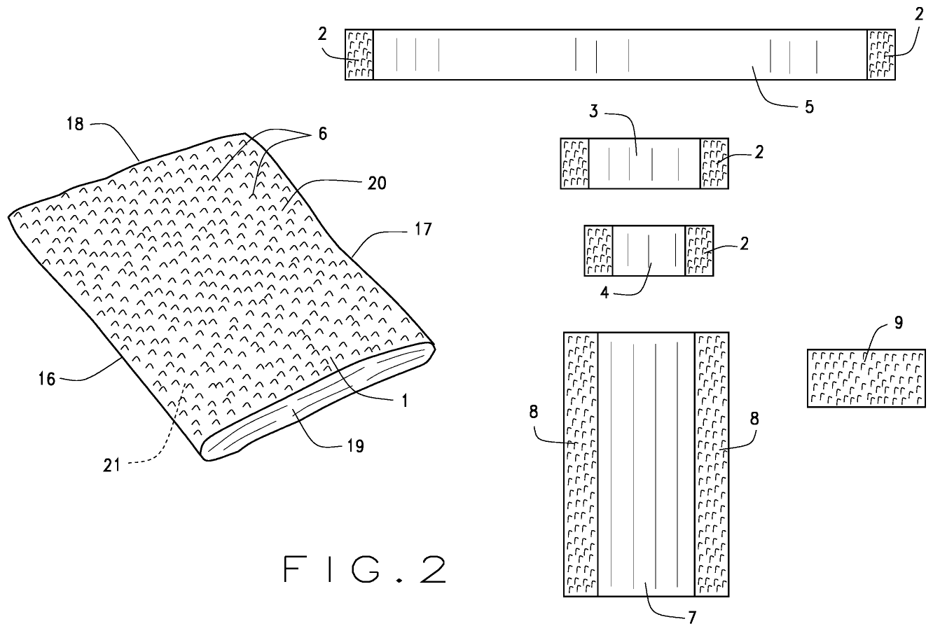

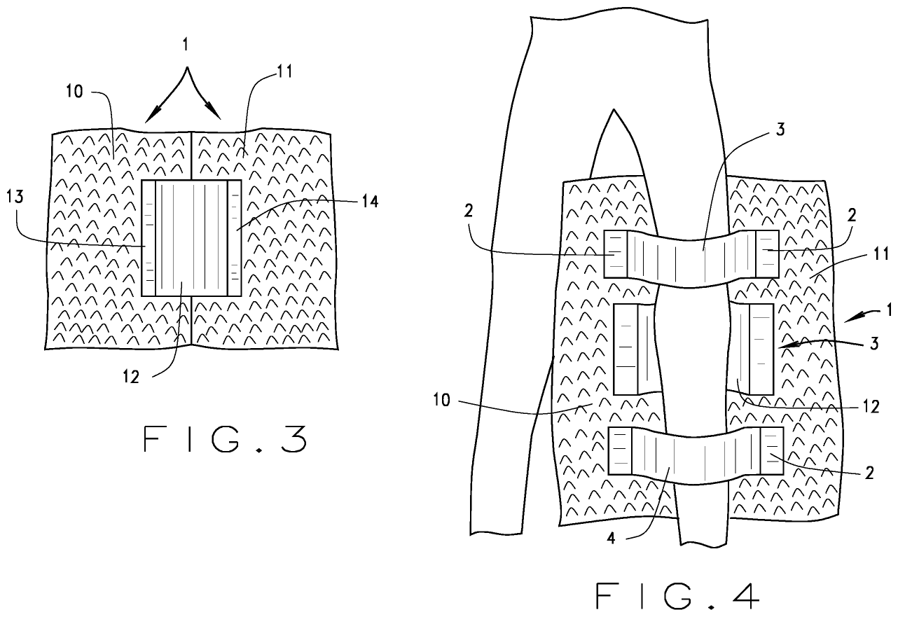

The various components that make up the securement of the patient to the pillow, as explained in FIG. 1, can be seen in FIG. 2. As noted, the pillow 1 can even have one of the hook and loop fastening means applied over the entire surface, as noted at 6, or the securement can be in the manner as previously described, with respect to the fasteners previously noted at 2. The various short straps 3 and 4, as can be seen, do have their hook and loop fastener means applied at their ends, as noted at 2, and these are for applications to the surface of a pillow, and to hold the arm in place, during rest. In addition, the back strap 5 can be seen, it also includes the use of the hook and loop fasteners, as at 2, as noted. In addition, in a lengthier or wider form of strapping mechanism can be seen at 7, having hook and loop fasteners to either side, as noted at 8, and this is provided for securement over a greater surface of the arm, as this lengthier or wider strap is applied to hold the arm in place, against the pillow 1. As can be noted, the various straps can be of elastic material, as shown at 3, 4 and 7, in order to add to the convenience of the application and securement of the arm to the pillow, during usage. In addition, the various straps, one is noted at 9, could actually have Velcro brand hook and loop type fasteners applied to its entire surface, in order to cooperate conveniently with its adherence to the pillow surface, during its application.

Where the extent of the injury undertaken healing, may require more than a single pillow or cushion in its application, such can be seen in FIG. 3. As noted, a pair of pillows 10 and 11 may be held together by a further connecting strap, as at 12, with the connecting strap 12 having hook and loop fasteners provided at either side edge, as at 13 and 14, in order to secure the pillows together. Then, these pillows or cushions may be wrapped around the leg, in a manner as to be subsequently described. Once again, the hook and loop type fasteners 13 and 14 may secure the isolated parts or opposite edges of the strap 12 to the pillow surface, or the entire connecting strap may be formed having a surface of hook and loop type fasteners, and the pillow surface having a corresponding surface of the other part of the hook and loop type fastener, in order to secure the two pillows together through the use of the connecting strap 12, as can be noted.

Where such a pillow or cushion arrangement is required for application to the leg of the patient, as can be noted in FIG. 4, the pair of pillows or cushions 10 and 11 held together by its connecting strap 12 can be applied to the leg, and then similar straps, such as straps 3 and 4 as previously reviewed, can be applied through their hook and loop type fasteners, as noted at 2, by securement of the arranged pillows to the leg of the patient, in an effort to cushion the leg during convalescence, such as when the patient locates upon the bed, either during resting, or while sleeping.

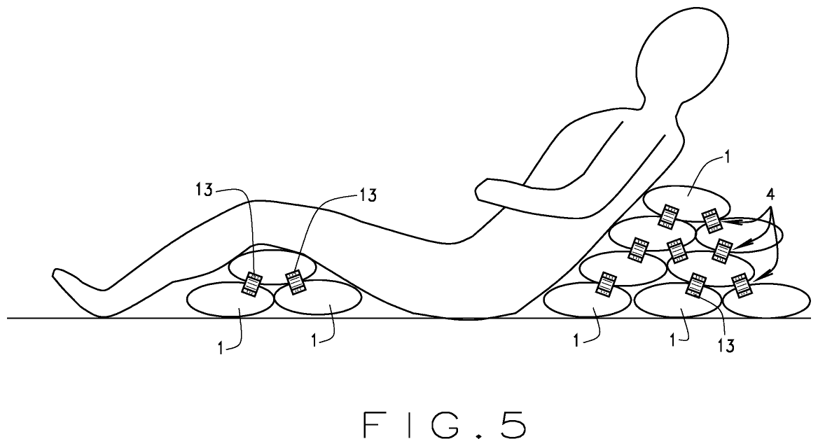

FIG. 5 shows how the various pillows, as noted at 1, can be secured together, by the hook and loop type fastening straps, as noted at 13, for securing the various pillows or cushions together, and to build up their volume, for providing cushioning either under the legs, as can be noted, or behind the back, as further can be noted in said figure, as a post-operative type of cushioning to add the comfort of the patient during convalescence. As an alternative, the various pillows or cushions 1 may have a surface texture of a very short type of loop or loop portion of the fastening means, as known as Velcro brand type fasteners or their equivalent, and the desired soft surface texture of the shown pillows or cushions may be applied to one or both surfaces, with the loop portion applied to the surface of the other pillow or cushion, in order to provide for securement of the pillows or cushions together, where strapping may not be needed. This can be a further means for nesting of the various pillows or cushions together, and to build them up into the required volume needed to elevate the leg, or comfort the back, to the extent that the patient requires, in order to obtain the type of cushioning, and comfort, during convalescence. As noted, a combination of three pillows or cushions may be applied under the knees of the patient, to add to the comfort at that location. And, the combination of pillows or cushions, can also be applied behind the back of the patient, or under the upper torso, and the head area of the patient, as noted, to add to the comfort, and support that part of the patient, during such convalescence, as for example after back surgery.

These are examples of how various combinations of the pillows or cushions, that may have a partial or full surface area of one component of the hook and loop type fastening means, while the various strappings, for their use may have the other of the hook and loop portion of the Velcro brand type fasteners or their equivalent, for example zippers, in order to secure the pillows or cushions together, and adhere them around the affected and repairing areas of the body, or the pillows may be looped together, through the application of their Velcro brand type fasteners or their equivalents, or held together by double sided strapping, for location under the various portions of the patient's body, to add to the support and comfort of the patient, during the healing process. And, as previously explained, the strapping could be applied to the pillows surface, and isolated locations, through the use of double sided tape at the area where Velcro brand type fasteners or their equivalent are to be applied, in order to allow for the straps to be adhered in place, about the body of the patient.

Thus, the cushioning system of this disclosure is comprised of the pillow or cushion 1 and the plurality of or combination of various different size straps 3, 4, 5 7, 9. The cushioning system in addition to the component parts described above could also be comprised of a further pair of pillows or cushions 10, 11 and their connecting strap 12. All of the cushions 1, 10, 11 and all of the straps 3, 4, 5, 7, 9, 12 are separate component parts of the cushion system. This provides for the versatility of the system. All of the component parts of the system are constructed of a flexible material, for example cloth or another equivalent type of material.

As represented in FIGS. 1 and 2, the cushion 1 has a rectangular configuration defined by opposite side edges 16, 17 and opposite end edges 18, 19. The cushion could have other, equivalent configurations. The four edges 16, 17, 18, 19 separate the first surface 20 on the first side of the cushion 1 from the second surface 21 on the second side of the cushion, where the second side is opposite the first side of the cushion. As represented in FIGS. 1 and 2, the entire first surface 20 of the cushion 1 is covered by one component of the hook and loop fastener. The opposite second surface 21 of the cushion 1 does not have either the first or second component of the hook and loop fastener on the surface. This presents the second surface 21 as a smooth and comfortable surface to engage against a body portion of a user of the cushion 1. In alternative embodiments, less than the entire first surface 20 of the cushion 1 could be provided with a component of the hook and loop fastener on the surface, and at least some portion of the second surface 21 could be provided with a component of the hook and loop fastener.

As represented in FIG. 2, the first surface 20 secured around the peripheral edges 16, 17, 18, 19 of the cushion 1 to the second surface 21 defines an interior volume of the cushion 1 between the first surface 20 and the second surface 21. The interior volume is filled with a soft material that provides the cushioning function of the cushion 1. In alternate embodiments, the interior volume of the cushion 1 could be empty and the cushion would have the configuration of a flat, flexible sheet.

Also as represented in FIGS. 1 and 2, each of the straps 3, 4, 5, 7 has a length with opposite first and second end portions of the strap length being separated by an intermediate portion of the length. For example, the strap 5 represented in FIG. 2 has a length with a first end portion at the left end of the strap 5 represented in FIG. 2 and a second end portion at the right end of the strap 5 represented in FIG. 2 being separated by an intermediate portion of the length of the strap. The first end portion 2 at the left end of the strap 5 has one component of the hook and loop fastener that is attachable on the first surface 20 of the cushion 1 and the second end portion 2 at the right end of the strap 5 has the one component of the hook and loop fastener that is attachable on the first surface 20 of the cushion 1. The opposite end portions 2 of the strap 5 releasably attach directly on the first surface 20 of the cushion and no separate fastener devices are needed to attach the strap 5 to the cushion 1. The intermediate portion of the strap 5 does not include a component of the hook and loop fastener and is not releasably attachable on the first surface 20 of the cushion 1. This provides the intermediate portion of the length of the strap 5 with a smooth comfortable surface for engagement against a body portion of a user of the system.

It should be noted from FIG. 2 that the other straps 3, 4, 7 are constructed in a similar manner to that of the strap 5 with an exception being that each of the straps 3, 4, 5, 7 has a length dimension that is different. This provides for the versatility of the system.

As represented in FIG. 3, each of the pillows or cushions 10, 11 of the pair represented has a first surface that is entirely covered by a component of a hook and loop fastener. Also as represented, the strap 12 has components of a hook and loop fastener only on the opposite end portions of the strap. The portions of the hook and loop fastener on the opposite ends of the strap 12 attach to the portions of the hook and loop fastener on the surfaces of the pair of cushions 10, 11 and thereby releasably attach the pair of cushions 10, 11 together.

Variations or modifications to the subject matter of this invention may occur to those skilled in the art on review of the disclosure provided herein. Such variations are within the spirit of the invention, and are intended to be encompassed within the scope of any claims to patent protection issuing upon this development. The various descriptions of the components of the invention, it's depiction of the drawings, and detailed explanation of the specifications, are primarily set forth for illustrative purposes only.

1. A cushioning device, such as a pillow, having one segment of a hook and loop fastening device connected to a portion or all of its exposed surfaces, and a strap having the other of the hook and loop fastening device secured thereto, so that the strap may be applied around the limb of a convalescing patient and securing with a hook and loop fasteners on the surface of the pillow to hold the pillow contiguously on the affected limb of the patient during healing.

2. The cushioning device of claim 1 wherein the portion of said hook and loop fastening device can be applied over the entire one side surface of the pillow, to add to the convenience of the application of the strap at various locations to secure the pillow upon the limb of the affected patient.

3. The cushioning device of claim 2, wherein said portion of the hook and loop fastening means is applied to both surfaces of the said pillow.

4. The cushioning device of claim 1, where there are a plurality of pillows, each having one portion of the hook and loop fastening means secured to a part of its surface, and a strap having the other of the hook and loop fastening means secured to both of its surfaces in order to secure a plurality of pillows together in preparation for usage and support of the patient during convalescence.

5. The cushioning device of claim 1, wherein there are a plurality of pillows, and said straps provided for securement of the plurality of pillows together for application in support of a patient during convalescence.

6. The cushioning device of claim 1, and including a zipper mechanism for attachment of a pair of pillows together.

7. A cushioning device, such as a plurality of pillows, each pillow having one segment of the hook and loop fastening device connected to a portion or all of its disclosed surfaces, and another pillow having the other segment of the hook and loop fastening device connected to a portion or all of its exposed surfaces, so that one pillow may be applied and adhered to the other pillow to secure them together for application in support of a portion of the patient during convalescence.

8. A cushioning system comprising:
a cushion, the cushion having a first surface on a first side of the cushion and a second surface on a second side of the cushion that is opposite the first side of the cushion;
a strap, the strap being separate from the cushion, the strap being releasably attachable on the first surface of the cushion.

9. The cushioning system of claim 8, further comprising:
the strap having a length, the length of the strap having first and second opposite end portions separated by an intermediate portion of the length of the strap; and
the first and second opposite end portions of the length of the strap being releasably attachable on the first surface of the cushion and the intermediate portion of the length of the strap not being releasably attachable to the cushion.

10. The cushioning system of claim 8, further comprising:
the strap being a first strap; and
a second strap, the second strap being separate from the cushion and separate from the first strap, the second strap being releasably attachable on the first surface of the cushion.

11. The cushioning system of claim 10, further comprising:
the cushion having an interior volume between the first surface and the second surface of the cushion; and
the interior volume of the cushion being filled with a soft material.

12. The cushioning system of claim 11, further comprising:
a third strap, the third strap being separate from the cushion and separate from the first strap and the second strap, the third strap being releasably attachable on the first surface of the cushion.

13. The cushioning system of claim 12, further comprising:
the cushion being a first cushion;
a second cushion, the second cushion being separate from the first cushion and separate from the first strap, the second strap and the third strap, the second cushion being releasably attachable to the first cushion, the first strap, the second strap and the third strap.

14. The cushioning system of claim 8, further comprising:
a first part of a hook and loop fastener on the first surface of the cushion; and
a second part of the hook and loop fastener on the strap, the second part of the hook and loop fastener being releasably attachable to the first part of the hook and loop fastener to releasably attach the strap on the first surface of the cushion.

15. The cushioning system of claim 14, further comprising:
the strap having a length with opposite end portions separated by an intermediate portion of the length of the strap; and
the second part of the hook and loop fastener being on the opposite end portions of the length of the strap and not on the intermediate portion of the length of the strap.

16. The cushioning system of claim 15, further comprising:
the strap being a first strap;
a second strap, the second strap being separate from the cushion and separate from the first strap; and
the second part of the hook and loop fastener being on the second strap, the second part of a hook and loop fastener being releasably attachable to the first part of the hook and loop fastener to releasably attach the second strap on the first surface of a cushion.

17. The cushioning system of claim 16, further comprising:
the first strap having a first length;
the second strap having a second length; and
the first length and the second length being different lengths.

18. The cushioning system of claim 16, further comprising:
a third strap, the third strap being separate from the cushion and separate from the first strap and the second strap; and
the second part of the hook and loop fastener being on the third strap, the second part of the hook and loop fastener on the third strap being releasably attachable to the first part of the hook and loop fastener on the first surface of the cushion to releasably attach the third strap on the first surface of the cushion.

19. The cushioning system of claim 14, further comprising:
the cushion being a first cushion;
a second cushion, the second cushion being separate from the first cushion;
the first part of the hook and loop fastener being on the second cushion; and
the second part of the hook and loop fastener on the strap being releasably attachable to the first part of the hook and loop fastener on the second cushion to releasably attach the strap on the second cushion.

20. The cushioning system of claim 19, further comprising:
the second part of the hook and loop fastener on the strap being releasably attachable to the first part of the hook and loop fastener on the first surface of the first cushion and to the second cushion to releasably attach the first cushion to the second cushion.
